(19) United States
(12) Patent Application Publication
(10) Pub. No.: US 2006/0286616 A1
(43) Pub. Date: Dec. 21, 2006

(54) Menopause stage monitor

(76) Inventors: Miki Furukawa, Ibaraki-shi (JP); Hitoko Matsunaga, Ibaraki-shi (JP); Mihoko Kawasaki, Ibaraki-shi (JP)

Correspondence Address: KNOBBE MARTENS OLSON & BEAR LLP, 2040 MAIN STREET, FOURTEENTH FLOOR, IRVINE, CA, 92614, US

(21) Appl. No.: 11/385,589

(22) Filed: Mar. 21, 2006

Related U.S. Application Data
(60) Provisional application No. 60/664,024, filed on Mar. 22, 2005.

(51) Int. Cl. G01N 33/53

(52) U.S. Cl. 435/7.92

(57) ABSTRACT

The present invention provides a portable menopausal phase monitor with which subjects can learn if they are menopausal, and if so, which phase they are at by using a body fluid such as urine, blood, or the like, as a test sample. A menopausal phase monitor of the present invention may have the following configuration: an absorbent member capable of migrating a sample by capillary action, the absorbent member composed of (a) a sample application zone for receiving and absorbing a sample; (b) an FSH reaction site carrying labeled anti-follicle stimulating hormone (FSH) antibodies, and an LH reaction site carrying labeled anti-luteinizing hormone (LH) antibodies; (c-1) an FSH detection zone provided with at least two FSH control displays with unlabeled anti-(anti-FSH antibody) antibodies immobilized thereon, and an FSH test result display with unlabeled anti-FSH antibodies immobilized thereon disposed at intervals, (c-2) an LH detection zone provided with at least two LH control displays with unlabeled anti-(anti-LH antibody) antibodies immobilized thereon, and an LH test result display with unlabeled anti-LH control display with unlabeled anti-(anti-LH antibody) antibodies immobilized thereon disposed at intervals; and (d) a sample collection zone for collecting residual sample that has migrated.

### BACKGROUND OF THE INVENTION

The present invention relates to menopausal phase monitors. More specifically, the present invention relates to portable menopausal phase monitors capable of easily identifying female menopausal phases using body fluids such as urine, blood, and the like as samples. The menopausal phase monitors enable women to easily determine themselves as necessary if they are in menopausal transition or not, and to identify their menopausal phases without visiting healthcare professionals.

### PRIOR ART

There are five stages across a women's lifespan, i.e. “childhood”, “adolescence”, “maturity”, “menopausal transition” and “senescence”, based on female hormone levels (estrogen=follicular hormone) produced by the ovaries. “Menopausal transition” begins at about age 40 when ovary endocrine functions gradually decline and ovulation frequency gradually decreases. Menstruation then gradually ceases and ovarian functions decrease as “menopausal transition” leads to “senescence”. Typically, “menopausal transition” refers to a period of several to ten years before and after perimenopause, and the internationally recognized menopausal ages are from 41 to 60.

Since the ovarian endocrine functions slow down and wane in the menopausal transition, endocrine imbalance and autonomic ataxia occur and cause palpitation, hot flashes, headaches, nausea, hyperesthesia, depression, unexplained fatigue, irritability, and like mental and physical discomforts (complaints). Such discomforts are called “menopausal discomforts”. Although hormonotherapy is required in some severe cases, most women can usually manage to lead normal lives. For example, women are able to handle mood swings experienced during the menopausal transition if they acknowledge that such changes are not illnesses but menopausal complaints caused by normal physiology during such a transition. For this reason, if women in their late thirties and older can learn by themselves if they are menopausal or not, or identify a menopausal phase they are at, they are able to adjust their daily routine in conformity with their hormonal balances under the awareness of being menopausal, and consequently cope well with the long menopausal transition.

Given this, self-assessment methods by which women apply a urine sample to a test strip and read the results by themselves have been proposed, and devices therefore, for easily detecting if one is menopausal (e.g. US Unexamined Patent Publication: US 2004/0063219 A1), and a home use test kit, “Estroven®—Menopause Monitor: Amerifit Nutrition, Inc.” (http://www.estrovenmonitor.com) is available in the United States. However, methods or devices for detecting each phase of the menopausal transition are not known.

### DISCLOSURE OF THE INVENTION

The object of the present invention is to provide portable menopausal phase monitors for subjects to identify if they are menopausal, or the phase of the menopausal transition the subjects are at by using their own body fluids such as urine, blood, and the like as test samples.

To solve the above problems, the present inventors conducted various kinds of research paying attention to hormonal fluctuations that occur during the menopausal transition, and found that production levels of follicle stimulating hormone (also simply referred to as “FSH” in the invention) and luteinizing hormone (also simply referred to as “LH” in the invention) secreted in blood and urine respectively vary depending on the phase in the menopausal transition. Based on these findings, the present inventors realized that the above object can be attained by monitors equipped with a detection zone capable of detecting, distinguishing and displaying such changes in these hormone secretion levels. As a result of further study, the present inventors accomplished two types of menopausal phase monitors below.

One of the types is a menopausal phase monitor (type 1) to detect the level of at least one of FSH and LH in a test sample, and comprises at least two control displays in addition to a test result display in a detection area. The other type is a menopausal phase monitor (type 2) comprising at least two reaction sites for FSH and LH respectively in a reaction zone and at least two detection zones, for FSH and LH respectively, in a detection area for simultaneous detection of FSH and LH in a sample to identify menopausal phases.

<Type 1>

Type 1 menopausal phase monitors specifically encompass the following configurations.

1-1. A menopausal phase monitor provided with an absorbent member composed of a material capable of migrating a test sample by capillary action, wherein the absorbent member comprises:
(1a) a sample application zone for receiving and absorbing a test sample;
(1b) an FSH reaction site carrying labeled anti-FSH antibody specifically reactive with follicle stimulating hormone (FSH);
(1c) an FSH detection zone equipped with at least two FSH control displays each with a different amount of immobilized antibody, and an FSH test result display disposed at intervals,
   (1) the FSH test result display having unlabeled anti-FSH antibody specifically reactive with follicle stimulating hormone (FSH) immobilized thereon, and
   (2) the FSH control displays having unlabeled anti-(anti-FSH antibody) antibody specifically reactive with anti-FSH antibody immobilized thereon; and
(1d) a sample collection zone for collecting residual sample that has migrated from the sample application zone, the FSH reaction site, and the FSH detection zone.

Using the above-mentioned menopausal phase monitor targeting follicle stimulating hormone (FSH) as a test substance and equipped with at least two FSH control displays with unlabeled anti-(anti-FSH antibody) antibodies indifferent concentrations immobilized thereon, at least three phases (Phase A, Phase B to D, and Phase E) of the menopausal transition of a subject can be detected and distinguished. The definitions of the phases of the menopausal transition used in the present invention are described later.

1-2. A menopausal phase monitor provided with an absorbent member composed of a material capable of migrating a test sample by capillary action, wherein the absorbent member comprises:
(2a) a sample application zone for receiving and absorbing a test sample;
(2b) an LH reaction site carrying labeled anti-LH antibody specifically reactive with luteinizing hormone (LH);
(2c) an LH detection zone equipped with at least two LH control displays with a different amount of immobilized antibody, and an LH test result display disposed at intervals,
   (1) the LH test result display having unlabeled anti-LH antibody specifically reactive with luteinizing hormone (LH) immobilized thereon, and
   (2) the LH control displays having unlabeled anti-(anti-LH antibody) antibody specifically reactive with anti-LH antibody immobilized thereon; and
(2d) a sample collection zone for collecting residual sample that has migrated from the sample application zone, the LH reaction site, and the LH detection zone.

Using the above-mentioned menopausal phase monitor targeting luteinizing hormone (LH) as a test substance and equipped with at least two LH control displays with unlabeled anti-(anti-LH antibody) antibodies in different concentrations immobilized thereon, at least three phases (Phase A to B, Phase C to D, and Phase E) of the menopausal transition of a subject can be detected and distinguished.

Type 1 menopausal phase monitors further encompass a menopausal phase monitor capable of simultaneously detecting both FSH and LH in a test sample as described below (this monitor can also be a Type 2 menopausal phase monitor described later).

1-3. A menopausal phase monitor provided with an absorbent member composed of a material capable of migrating a test sample by capillary action, wherein the absorbent member comprises:
(3a) a sample application zone for receiving and absorbing a test sample;
(3b) a reaction zone comprising an FSH reaction site carrying labeled anti-FSH antibody specifically reactive with follicle stimulating hormone (FSH), and an LH reaction site carrying labeled anti-LH antibody specifically reactive with luteinizing hormone (LH);
(3c) a detection area comprising an FSH detection zone and an LH detection zone,
   (3c-1) the FSH detection zone being equipped with at least two FSH control displays each with a different amount of immobilized antibody, and an FSH test result display disposed at intervals,
   (i) the FSH test result display having unlabeled anti-FSH antibody specifically reactive with follicle stimulating hormone (FSH) immobilized thereon,
   (ii) the FSH control displays having unlabeled anti-(anti-FSH antibody) antibody specifically reactive with anti-FSH antibody immobilized thereon,
   (3c-2) the LH detection zone being equipped with at least two LH control displays each with a different amount of immobilized antibody, and an LH test result display disposed at intervals,
   (i) the LH test result display having unlabeled anti-LH antibody specifically reactive with luteinizing hormone (LH) immobilized thereon, and
   (ii) the LH control displays having unlabeled anti-(anti-LH antibody) antibody specifically reactive with anti-LH antibody immobilized thereon; and
(3d) a sample collection zone for collecting residual sample that has migrated from the sample application zone, the reaction zone, and detection area.

Using the above-mentioned menopausal phase monitor targeting follicle stimulating hormone (FSH) and luteinizing hormone (LH) as test substances, and equipped with at least two FSH control displays with unlabeled anti-(anti-FSH antibody) antibodies in different concentrations immobilized at the detection zone for the test substance FSH in addition to an FSH test result display as well as at least two LH control displays with unlabeled anti-(anti-LH antibody) antibodies in different concentrations immobilized at the detection zone for the test substance LH in addition to an LH test result display, at least four phases (Phase A, Phase B, Phase C to D, and Phase E) of the menopausal transition of a subject can be detected and distinguished.

<Type 2>

Type 2 menopausal phase monitors specifically have the following configurations:

2-1. A menopausal phase monitor provided with an absorbent member composed of a material capable of migrating a test sample by capillary action, wherein the absorbent member comprises:
(4a) a sample application zone for receiving and absorbing a test sample;
(4b) an FSH reaction site carrying labeled anti-FSH antibody specifically reactive with follicle stimulating hormone (FSH), and an LH reaction site carrying labeled anti-LH antibody specifically reactive with luteinizing hormone (LH);
(4c-1) an FSH detection zone equipped with an FSH control display, and an FSH test result display disposed at intervals,
   (1-1) the FSH test result display having unlabeled anti-FSH antibody specifically reactive with follicle stimulating hormone (FSH) immobilized thereon, and
   (1-2) the FSH control display having unlabeled anti-(anti-FSH antibody) antibody specifically reactive with anti-FSH antibody immobilized thereon;
(4c-2) an LH detection zone equipped with an LH control display, and an LH test result display disposed at intervals,
   (2-1) the LH test result display having unlabeled anti-LH antibody specifically reactive with LH immobilized thereon, and
   (2-2) the LH control display having unlabeled anti-(anti-LH antibody) antibody specifically reactive with anti-LH antibody immobilized thereon; and
(4d) a sample collection zone for collecting residual sample that has migrated from the sample application zone, the FSH reaction site, the LH reaction site, the FSH detection zone, and the LH detection zone.

Using the above menopausal phase monitor which targets follicle stimulating hormone (FSH) and luteinizing hormone (LH) as test substances, at least three phases (Phase A, Phase B, and Phase C to E) of the menopausal transition of a subject can be detected and distinguished.

Type 2 menopausal phase monitors further encompass, as described below, menopausal phase monitors equipped with at least two FSH control displays with unlabeled anti-(anti-FSH antibody) antibodies in different concentrations immobilized at an FSH detection zone in addition to an FSH test result display, and at least two LH control displays with unlabeled anti-(anti-LH antibody) antibodies in different concentrations immobilized at an LH detection zone in addition to an LH test result display (this monitor can also be a Type 1 menopausal phase monitor described above).

2-2. A menopausal phase monitor provided with an absorbent member composed of a material capable of migrating a sample by capillary action, wherein the absorbent member comprises:
(5a) a sample application zone for receiving and absorbing a sample;
(5b) an FSH reaction site carrying labeled anti-FSH antibody specifically reactive with follicle stimulating hormone (FSH), and an LH reaction site carrying labeled anti-LH antibody specifically reactive with luteinizing hormone (LH);
(5c-1) an FSH detection zone equipped with at least two FSH control displays each with a different amount of immobilized antibody, and an FSH test result display disposed at intervals,
   (1-1) the FSH test result display having unlabeled anti-FSH antibody specifically reactive with follicle stimulating hormone (FSH) immobilized thereon, and
   (1-2) the FSH control displays having unlabeled anti-(anti-FSH antibody) antibody specifically reactive with anti-FSH antibody immobilized thereon;
(5c-2) an LH detection zone equipped with at least two LH control displays each with a different amount of immobilized antibody, and an LH test result display disposed at intervals,
   (2-1) the LH test result display having unlabeled anti-LH antibody specifically reactive with luteinizing hormone (LH) immobilized thereon, and
   (2-2) the LH control displays having unlabeled anti-(anti-LH antibody) antibody specifically reactive with anti-LH antibody immobilized thereon; and
(5d) a sample collection zone for collecting residual sample that has migrated from the sample application zone, the FSH reaction site, the LH reaction site, the FSH detection zone, and the LH detection zone.

Using the above-mentioned menopausal phase monitor which targets two test substances, follicle stimulating hormone (FSH) and luteinizing hormone (LH), and is equipped with at least two FSH control displays each with unlabeled anti-(anti-FSH antibody) antibodies of different concentrations immobilized at FSH detective zone, and with at least two LH control displays each with unlabeled anti-(anti-LH antibody) antibodies of different concentrations immobilized at LH detection zone, at least four phases (Phase A, Phase B, Phase C to D, and Phase E) of the menopausal transition a subject is at can be detected and distinguished.

The term “menopausal phases” used in the invention refer to the perimenopausal phases proposed by J C Prior, a Canadian endocrinologist (Prior J C, Endo Rev., 19, 397-428, (1988)). In this document, Prior defines the perimenopausal transition as “the period immediately before menopause when the endocrinological, biological and clinical signs of approaching menopause commence and at least the first year after menopause”, and categorizes the period into five phases, Phases A to E as shown in Table 1 below.

TABLE 1

| | Phase A | Phase B | Phase C | Phase D | Phase E |
|---|---|---|---|---|---|
| Duration | 2-6 months | 2-6 months | 1-2 years | 1-2 years | 1 year |
| Menstrual | Regular, | Regular, | Irregular, | Oligo- | Amenorrhea |
| cycles | Short | Luteal | Ovulation less | Amenorrhea, | |
| | follicular | dysfunction | than 50% | infrequent | |
| | phase | | | ovulation | |
| Menstrual | Increased or | Increased | Heavy or scant | Spotting | None |
| flow | normal | | (often | alternating | |
| | | | alternating) | with | |
| | | | | flooding | |
| Menstrual | PMS ↑ | PMS ↑↑ | PMS alleviated | Infrequent | None |
| cycle- | Dysmenorrhea, | (intermittent) | Occasional | menstrual- | |
| related | swollen | dysmenorrhea | menstrual- | like cramps | |
| symptoms | breasts, | | type cramps | | |
| | depression, | | | | |
| | irritability, | | | | |
| | exacerbation of | | | | |
| | headaches and | | | | |
| | migraines | | | | |
| Hormone | LH → | LH → | LH ↑ | LH ↑ (stable) | LH ↑↑ |
| levels in | FSH → | FSH ↑ | (occasionally) | FSH ↑ | FSH ↑↑ |
| serum | | (initial | FSH ↑ | | |
| | | phase of | (persistently) | | |
| | | follicular | | | |
| | | phase) | | | |

→: normal,
↑: slightly elevated,
↑↑: greately elevated
PMS: Premenstrual Syndromes

“Menopausal phases” used in the invention refer to the five menopausal phases (Phases A, B, C, D and E) according to J C Prior's definitions.

The Japan Society of Obstetrics and Gynecology defines “menopausal transition” as “a transitional period from the reproductive phase (sexually mature phase) to the non-reproductive phase (senescence), during which ovarian functions start declining and cease, and generally refers to a period of five years, from before to after the menopause”. However they are not different in essence.

Menopausal phase monitors (types 1 and 2) of the invention are made such that a subject can easily identify with the naked eye which of the aforementioned menopausal phases (Phases A to E) the subject is at by bringing a sample such as urine, blood, etc. to the monitor.

Menopausal phase monitors of the invention are equipped with an absorbent member made of materials capable of migrating a sample by capillary action or chromatographic action (these actions are collectively referred to as “capillary action” in the invention). Such an absorbent member may be in the form of sheet strip (hereinafter referred to as “sheet strip”), and such a sheet strip may be composed of a single sheet, or a plurality of sheets superposed on or connected to one another. Alternatively, the absorbent member may be in the form of a long rod or in any of various forms capable of absorbing a fluid and transporting it by capillary action. Menopausal phase monitors of the invention may further be equipped with a support for supporting the absorbent member.

Materials for composing the absorbent member are not limited, and may be any material having a porous or capillary structure in which solvents (water, urine, and other body fluids), test substances (follicle stimulating hormone (FSH) and luteinizing hormone (LH)), antibodies specific to such test substances (e.g. labeled anti-FSH antibody, and labeled anti-LH antibody), and complexes containing such test substances (e.g. a complex between labeled anti-FSH antibody and FSH, and a complex between labeled anti-LH antibody and LH) can be permeated, and by which a test substance-containing sample can pass through (develop) together with such above antibodies and complexes when applied thereto (received). Examples of such materials include filter papers, chromatography papers and like sheets; felts, woven fabrics, non-woven fabrics, and like fabrics; glass fibers, etc. Among these, organic porous materials are preferable. Examples of organic porous materials include fiber assemblies made from cellulose and like natural fibers; cellulose acetate and like semi-synthetic fibers; nitrocellulose and like cellulose derivatives; polyethylenes, polypropylenes, nylons, polyesters and like synthetic fibers; porous polypropyelenes, porous polystyrenes, porous polymethacrylate methyl, porous nylons, porous polysulfones, porous fluoroplastics, and hydrophilic group-introduced polyvinylidene fluorides and like porous synthetic resins. Preferable are cellulose and like natural fibers, cellulose acetate and like semi-synthetic fibers, and nitro cellulose and like cellulose derivatives.

The absorbent member is not limited in size, and is sized about 2 to about 30 mm in width (length of the shorter side), and preferably about 5 to about 15 mm, and about 5 to about 200 mm in length (length of a longer side), and preferably about 10 to about 150 mm.

### BRIEF DESCRIPTION OF DRAWINGS

FIG. 1 is a perspective view showing an embodiment of a Type 1 menopausal phase monitor (1) of the invention. In the figure, A is a sample application zone, B is a reaction zone, C is a detection area, D is a sample collection zone, C0 is an FSH test result display, C1 and C2 are FSH control displays, 10 is a support, 20 is an absorbent member (sheet strip), 21 is a sheet that is a component of the sample application zone (A), 22 is a sheet that is a component of the detection area (C), 23 is a sheet that is a component of the sample collection zone (D), and 24 is a sheet that is a component of the reaction zone (B). Further, the arrow p shows the sample migration direction (hereinafter the same numbers refer to equivalent parts in the following figures).

FIG. 2 is a perspective view showing an embodiment of a Type 2 menopausal phase monitor (2) of the invention. In this figure, 30 is an absorbent member (sheet strip), b is an FSH reaction site, b′ is an LH reaction site, c0 is an FSH test result display, c1 and c2 are FSH control displays, c0′ is an LH test result display, c1′ and c2′ are LH control displays, 31 is a sheet that is a component of the sample application zone (A), 32 and 33 are sheets that are components of the detection area (C), 35 is a sheet that is a component of the sample collection zone (D), and 34 is a gap.

FIG. 3 is a perspective view showing another embodiment of a Type 2 menopausal phase monitor of the invention. In this figure, 40, 41 and 42 are absorbent members (sheet strips).

FIG. 4 is a perspective view showing another embodiment of a Type 2 menopausal phase monitor of the invention. In this figure, 50 and 51 are absorbent members (sheet strips), 54 is a gap, 52 and 53 are each respective side of the sheet strip separated by the gap, and 55 and 56 are each respective end in the length ways direction of the sheet strip.

FIG. 5 is a perspective view showing another embodiment of a Type 1 menopausal phase monitor of the invention. In this figure, 60 to 62 are absorbent members (sheet strips), and of these 61 is a wider sheet strip portion.

FIG. 6 is a perspective view showing another embodiment of a Type 1 menopausal phase monitor of the invention. 70 is a case, 71 is a case body, 72 is a case lid, 73 to 75 are windows, 76 is a slot, and 77 is a button slidable in the slot.

FIG. 7 is a perspective view showing another embodiment of a Type 2 menopausal phase monitor of the invention. In this figure, 80 is a case, 81 is a case body, 82 is a case cap, and 83 to 85 and 83′ to 85′ are windows.

FIG. 8 is a perspective view showing another embodiment of a Type 1 menopausal phase monitor of the invention. In this figure, 90 is a rod.

FIG. 9 is a perspective view showing another embodiment of a Type 1 menopausal phase monitor of the invention. In this figure, 102 is a circular cylindrical rod member of an absorbent member, and 101 and 103 are sheet members of the absorbent member.

FIG. 10 is a perspective view showing another embodiment of a Type 2 menopausal phase monitor of the invention. In this figure, 112 is a rectangular cylindrical rod of an absorbent member, and 111 and 113 are sheet members of the absorbent member.

### BEST MODE FOR CARRYING OUT THE INVENTION

Preferred embodiments of each type of menopausal phase monitors (Types 1 and 2) of the invention are described below with reference to the accompanying drawings.

(1) Type 1: Menopause Phase Monitor

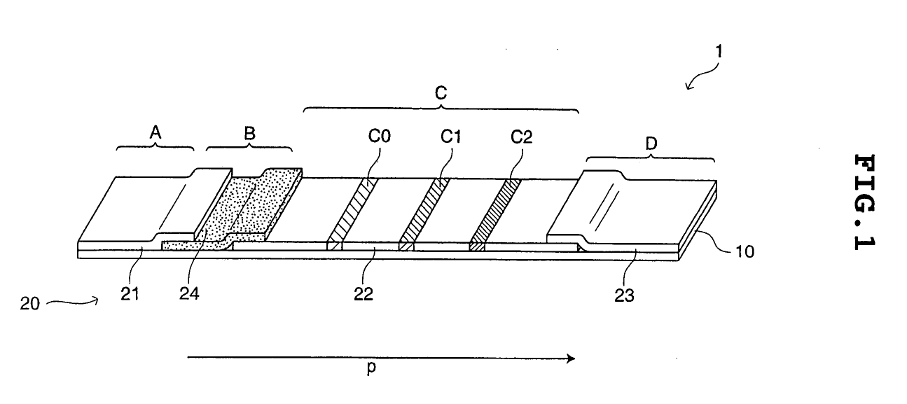

FIG. 1 shows one of the embodiments of a Type 1 menopausal phase monitor. FIG. 1 is a perspective view of a Type 1 menopausal phase monitor (hereinafter simply referred to as “menopausal phase monitor 1”). The menopausal phase monitor 1 is described below with reference to FIG. 1.

In menopausal phase monitor 1, an absorbent member is equipped with a sample application zone (A) for receiving a test sample, a reaction zone (B), a detection area (C), and a sample collection zone (D) for absorbing residual sample that has passed through the above application zone, reaction zone and detection area. In this embodiment, the absorbent member is a sheet strip composed of a plurality of sheets, but the sheet strip may be composed of a single sheet all of the same material, or may alternatively be composed of a single sheet consisting integrally of a plurality of sheets made from the same or different materials. Further, the sheet may be entirely composed of a plurality of sheets, or may be partially composed of a plurality of sheets.

In the embodiment shown in the figure, a sheet strip 20 is adhered on a support 10. The sheet strip 20 is equipped with a sheet 21 that is a component of the sample application zone (A), a sheet 22 that is a component of the detection area (C), and a sheet 23 that is a component of the sample collection zone (D) from one end to the other end in the lengthways direction respectively, wherein one end of each sheet 21 and 23 overlaps a different end of the sheet 22, thereby enabling a sample to migrate. Further, a sheet 24 that is a component of the reaction zone (B) is adhered underneath the sheet 21 at a position close to the sheet 22.

The sample application zone (A) is a component for receiving and absorbing a test sample for assaying (sample supplying component). Applicable test samples are not limited, as long as they contain a test substance for assaying of the invention [one or both of follicle stimulating hormone (FSH) and luteinizing hormone (LH)], such as urine, blood (serum, plasma), sweat, saliva, and like body fluids. For easy collection, a urine sample is preferable. As shown in FIG. 1, the sample application zone (A) is basically located at one end (upstream) of the sheet strip. This enables a subject to collect a test sample by dipping the end of the sheet strip in a pottle of urine. Alternatively, when using a urine sample, a subject can directly place the sample application zone in a urine stream while urinating.

Among materials for composing the sheet strip, the sample application zone (A) is preferably made of a hydrophilic and absorbent material so as to quickly absorb a test sample (body fluid such as urine, etc.). Examples of such materials are those made from various fibers [cotton, pulp, hemp, silk, wool, flax, and like natural fibers; rayon, polynosic, cupra, lyocell, and like regenerated fibers; acetates and like semi-synthetic fibers; polyamides (nylon), vinylons (polyvinylalcohols), polyesters, acrylics (polyacrylonitriles, etc.), polyolefins (polyethylenes, polypropylenes, etc.), polyurethanes, and like synthetic fibers; glass fibers], etc. such as filter papers, felts, nonwoven fabrics, woven fabrics, waterleaf papers, sponges, and the like. Preferable are nonwoven fabrics and woven fabrics, sponges, waterleaf papers, and the like made from polyesters, polypropylenes, rayons, and/or glass fibers.

The reaction zone (B) is located downstream of the sample application zone (A) as described earlier, and is on the sheet 24 partially overlapping the sample application zone (A). The reaction zone (B) contains detachable antibodies that specifically react and bind to a test substance (FSH or LH) for assaying.

More specifically, a menopausal phase monitor 1 targeting follicle stimulating hormone (FSH) as a test substance (substance for detection) (hereinafter simply referred to as “menopausal phase monitor 1 (FSH)” contains at the sheet 24 detachable anti-FSH antibodies that specifically bind to FSH by antigen-antibody reaction. Similarly, a menopausal phase monitor 1 targeting luteinizing hormone (LH) as a test substance (substance for detection) (hereinafter simply referred to as “menopausal phase monitor 1 (LH)” contains at the sheet 24 detachable anti-LH antibodies that specifically bind to LH by antigen-antibody reaction.

Desirable anti-FSH antibodies herein are those that specifically bind to FSH and do not cross-react with other proteins Such anti-FSH antibodies are commercially available in the form of anti-human follicle stimulating hormone antibody (murine) and the like from, for example, Biogenesis LTD., etc. Similarly, desirable anti-LH antibodies herein are those that specifically bind to LH and do not cross-react with other proteins. Such anti-LH antibodies are commercially available in the form of anti-human luteinizing hormone antibody (murine) and the like from, for example, Biogenesis LTD., etc.

These antibodies (anti-FSH antibodies and anti-LH antibodies) are used by being tagged with a labeling agent. Labeling agents usable herein include gold, silver, selenium, and like metals and inorganic particles; fluorescein, rhodamine, and like fluorescent labeling agents; red blood cells, latex particles, dyed or colored latex particles, and like pigmented labeling agents; β-galactosidase, alkali phosphatase, peroxidase, and like enzyme labeling agents; and mixtures thereof. Preferable are inorganic gold particles. Labeling methods using labeling agents are well known, and can be performed in a routine manner in accordance with the labeling agent used.

Among materials for composing the sheet strip, the reaction zone (B) is preferably made of a hydrophilic and absorbent material which contains labeled anti-FSH antibodies or labeled anti-LH antibodies in a detachable state, and has properties capable of transporting antigen-antibody complexes (FSH and labeled anti-FSH antibody complex, LH and labeled anti-LH antibody complex) formed by the reaction of the antibody and a test substance (FSH or LH) in a test sample arriving from the sample application zone (A) in the direction shown by the arrow p in the figures while such complexes continue to proceed to the sample detection area (C).

Examples of such materials those made from various fibers [cotton, pulp, hemp, silk, wool, flax, and like natural fibers; rayon, polynosic, cupra, lyocell, and like regenerated fibers; acetate and like semi-synthetic fibers; polyamides (nylon), vinylons (polyvinylalcohols), polyesters, acrylics (polyacrylonitrile, etc.), polyolefins (polyethylenes, polypropylenes, etc.), polyurethanes, and like synthetic fibers; glass fibers], etc. such as filter papers, felts, nonwoven fabrics, woven fabrics, waterleaf papers, sponges, and the like. Preferable are nonwoven fabrics and woven fabrics, sponges, waterleaf papers, and the like made from polyesters, polypropylenes, rayons, and/or glass fibers.

Methods for carrying labeled anti-FSH antibodies or labeled anti-LH antibodies in a detachable state on the reaction zone (B) are not limited, and examples include a method wherein the reacting zone (B) is impregnated with or adhered to by a solution containing labeled anti-FSH antibodies or labeled anti-LH antibodies. Further, it is preferable that such labeled anti-FSH antibodies or labeled anti-LH antibodies be carried in an excessive amount so that the test substance (FSH or LH) in a sample inevitably binds to such antibodies.

The detection area (C) further transports the sample, having passed through the above sample application zone (A) and reaction zone (B) to a sample collection zone (D), and has a portion of its sample transporting region such that a specific substance in the migrating sample is captured and the capturing result is displayed [test result display (C0)]. Menopausal phases become identifiable based on the results displayed at this test result display (C0), and the portion containing such a display (C0) is thereby termed “detection area (C)”.

The detection area (C) of a menopausal phase monitor 1 of the invention comprises at least two control displays (C1) and (C2) each with a different amount of immobilized antibodies, in addition to the test result display (C0). The test result display (C0) and two or more control displays (C1) and (C2) are spaced apart from one another.

Unlabeled antibodies [menopausal phase monitor 1 (FSH): unlabeled anti-FSH antibodies, menopausal phase monitor 1 (LH): unlabeled anti-LH antibodies] that specifically react with a test substance (FSH or LH) are immobilized in an excessive amount on the test result display (C0). A test substance bound to a labeled antibody by antigen-antibody reaction at the reaction zone (B) is captured at the test result display (C0), and develops color caused by a labeling agent at an intensity according to the amount captured. More specifically, in menopausal phase monitor 1 (FSH), FSH bound to labeled anti-FSH antibodies (labeled anti-FSH antibody—FSH complex) by antigen-antibody reaction at the reaction zone (B) is captured at the test result display (C0) on which unlabeled anti-FSH antibodies are immobilized, and develops color caused by a labeling agent at an intensity according to the amount captured. Similarly, in menopausal phase monitor 1 (LH), LH bound to labeled anti-LH antibody (labeled anti-LH antibody—LH complex) by antigen-antibody reaction at the reaction zone (B) is captured at the test result display (C0) on which unlabeled anti-LH antibodies are immobilized, and develops color caused by a labeling agent at an intensity according to the amount captured.

An amount of antibodies immobilized at the test result display (C0) is not restricted, and for example, for “menopausal phase monitor 1 (FSH)” using a blood sample, unlabeled anti-FSH antibodies may be immobilized at the test result display in such an amount that they bind to FSH, e.g. greater than 200 mIU/mL in blood. For “menopausal phase monitor 1 (LH)” using a blood sample, unlabeled anti-LH antibodies may be immobilized at the test result display in such an amount that they bind to LH, e.g. greater than 200 mIU/mL in blood. For a case using a urine sample, about 2 to about 10% of each of the above values can be considered as guideline values.

In the control displays (C1) and (C2), unlabeled antibodies (secondary antibodies) specifically reactive with the labeled antibodies contained at the above reaction zone (B) are immobilized in a certain amount. Menopausal phase monitors 1 of the invention have such control displays at at least two places in the detection area (C). More specifically, in the sample migration direction (direction p in the figures), the first control display (C1) is disposed at a distance from the test result display (C0), the second control display (C2) is similarly disposed at a distance from the first control display, and the nth control display (Cn) is similarly disposed . . . , and so on. Unlabeled antibodies (secondary antibodies) specifically reactive with labeled antibodies contained in a sample are immobilized in different amounts at each control display. It is preferable, but not restricted thereto, that secondary antibodies present in low concentrations are immobilized at the first control display (C1), and that antibodies of higher concentrations are immobilized at higher numbered control displays.

Labeled antibodies leaving the reaction zone and released into the sample are captured in the above control displays, and develop color caused by a labeled antibody labeling agent at intensities based on the amount of secondary antibodies immobilized at each control display.

More specifically, at each control display of the menopausal phase monitors 1 (FSH), unlabeled anti-(anti-FSH antibody) andibodies (secondary antibodies) specifically reactive with labeled anti-FSH antibodies are immobilized in a certain amount, and at each control display of the menopausal phase monitors 1 (LH), unlabeled anti-(anti-LH antibody) andibodies (secondary antibodies) specifically reactive with labeled anti-LH antibodies are immobilized in a certain amount.

Preferable anti-(anti-FSH antibody) antibodies herein are those that specifically bind to anti-FSH antibodies and do not crossreact with other proteins. Examples of such anti-(anti-FSH antibody) antibodies can be prepared in a routine manner using, for example anti-FSH antibodies (e.g. available from Biogenesis LTD.) Similarly, preferable anti-(anti-LH antibody) antibodies are those that specifically reactive with anti-LH antibodies and do not crossreact with other proteins. Such anti-(anti-LH antibody) antibodies can be prepared in a routine manner using, for example anti-LH antibodies (e.g. available from Biogenesis LTD.)

For example, a “menopausal phase monitor 1 (FSH)” using a blood sample preferably contains at least a control display (C1) on which anti-(anti-FSH antibody) antibodies (secondary antibodies) are immobilized so as to quantitatively detect an amout of anti-FSH antibodies that bind to FSH ranging from 0 to 50 mIU/mL, and preferably from 15 to 30 mIU/mL in a test sample, and a control display (C2) on which anti-(anti-FSH antibody) antibodies (secondary antibodies) are immobilized so as to quantitatively detect an amount of anti-FSH antibodies that bind to FSH ranging from 25 to 200 mIU/mL, and preferably from 40 to 100 mIU/mL in a test sample.

Further, for example, a “menopausal phase monitor 1 (LH)” using a blood sample preferably contains at least a control display (C1) on which anti-(anti-LH antibody) antibodies (secondary antibodies) are immobilized so as to quantitatively detect an amout of anti-LH antibodies that bind to LH ranging from 0 to 30 mIU/mL, and preferably from 5 to 20 mIU/mL in a sample, and a control display (C2) on which anti-(anti-LH antibody) antibodies (secondary antibodies) are immobilized so as to quantitatively detect an amount of anti-LH antibodies that bind to LH ranging from 15 to 200 mIU/mL, and preferably from 20 to 100 mIU/mL in a sample.

Menopausal phases can be identified based on a comparison between color intensities (tones) displayed at the above at least two control displays (C1) and (C2) and that of the above test result display (C0) of the reference area (C).

Among materials for composing the sheet strip, the detection area (C) is preferably made of a hydrophilic and absorbent material that has properties capable of migrating a sample containing various substances to the sample collection zone (D) by capillary action, and stably immobilizing the above unlabeled antibodies at the test result display (C0) as well as the above unlabeled secondary antibodies at the control displays (C1) and (C2).

Examples of such materials are those made from various fibers [cotton, pulp, hemp, silk, wool, flax, and like natural fibers; rayon, polynosic, cupra, lyocell, and like regenerated fibers; acetates and like semi-synthetic fibers; polyamides (nylon), vinylons (polyvinylalcohols), polyesters, acrylics (polyacrylonitrile), polyolefins (polyethylenes, polypropylenes, etc.), polyurethanes, and like synthetic fibers; glass fibers], etc. suh as filter papers, felts, nonwoven fabrics, woven fabrics, waterleaf papers, sponges, and the like. Preferable are nonwoven fabrics and woven fabrics, sponges, waterleaf papers, and the like made from cellulose and/or nitrocellulose, etc.

The sample collection zone (D) is a component for collecting residual sample that has migrated from the above sample application zone (A) to the reaction zone (B) and onto the detection area (C) (test result display (C0) and control displays (C1) and (C2)) in the direction of the arrow p.

Among materials for composing the sheet strip, the sample collection zone (D) is preferably made of a hydrophilic and absorbent material, and more preferably made of a material that does not release the absorbed residues or bulky material. Specific examples of such materials include absorbent cottons, hydrophilic fibers, and like nonwoven fabrics, and may include layered materials of absorbent cottons with nonwoven fabrics.

Menopausal phase monitors 1 targeting either follicle stimulating hormone (FSH) or luitenizing hormone (LH) as a test substance for assaying [menopausal phase monitor 1 (FSH) and menopausal phase monitor 1 (LH)] have been described above.

However, menopausal phase monitor 1 may concurrently target both follicle stimulating hormone (FSH) and luteinizing hormone (LH) as test substances to be assayed (hereinafter referred to as a “menopausal phase monitor 1 (FSH/LH). The menopausal phase monitor 1 (FSH/LH) comprises the “reaction zone (B)” located between the sample application zone (A) and the sample collection zone (D) equipped with an FSH reaction site (b) carrying labeled anti-FSH antibodies specifically reactive with follicle stimulating hormone (FSH), and an LH reaction site (b′) carrying labeled anti-LH antibodies specifically reactive with luteinizing hormone (LH) and the “detection area (C)” located immediately after the reaction sites equipped with:

(i) an FSH detection zone (c) equipped with at least two FSH control displays each with a different amount of immobilized antibodies, and an FSH test result display as below disposed at intervals,
(1) the FSH test result display (c0) being where unlabeled anti-FSH antibodies specifically reactive with FSH are immobilized, and
(2) the FSH control displays (c1) and (c2) being where unlabeled anti-(anti-FSH antibody) antibodies specifically reactive with anti-FSH antibodies are immobilized; and

(ii) an LH reference zone (c′) equipped with at least two LH control displays with a different amount of immobilized antibodies, and an LH test result display as below disposed at intervals,
(1) the LH test result display (c0′) being where unlabeled anti-LH antibodies specifically reactive with LH are immobilized, and
(2) the LH control displays (c1′) and (c2′) being where unlabeled anti-(anti-LH antibody) antibodies specifically reactive with anti-LH antibodies are immobilized.

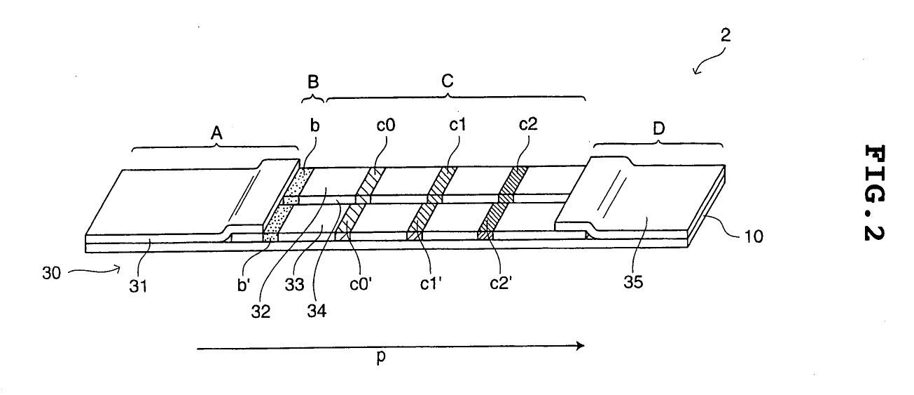

The positional relationships on the sheet strip between the FSH reaction site (b) and the LH reaction site (b′), and between the FSH detection zone (c) and the LH detection zone (c′), are not limited so long as FSH and LH levels in a test sample are assayed and the objects of the present invention are attained. As shown in FIG. 2, one of embodiments is a monitor provided with the detection area (C) comprising an FSH detection zone (c) and an LH detection zone (c′) separated by a gap.

Menopausal phase monitors 1 of the invention basically comprise a sheet strip equipped with the above components; however in addition to such a sheet strip, they may be further equipped with a support 10. A support 10 is not limited as long as it is made of material(s) and in a configuration capable of holding the above sheet strip. The support may be used, for example, in the form of a sheet laminated on the backside (underside) of the sheet strip, or as a housing configuration for encasing a sheet strip.

Examples of sheet supports include various sheets made of plastic (plastic sheets), hardened paper, aluminium sheets and like metal (including alloy) sheets, multi-layered papers of the same or different materials overlaying (adhered to) one another, those of plastic sheet(s) and paper overlaying (adhered to) one another, those of paper and metal sheet(s) overlaying (adhered to) one another, those of plastic sheet (s), paper and metal sheet(s) overlaying (adhered to) one another, papers coated for waterproofing and the like, etc. Preferable are those that are waterproof.

Preferable examples of support materials in the form of a housing configuration include polyvinyl chloride, polypropylene, polyethylene, polystyrene, acrylic acid polymers, and like moisture-impermeable materials. Such a housing configuration is desirably provided at least with a “sample application window” for a sample application zone (A), a “detection display window” for a test result display (C0) of the detection area (C), and “control display windows” for the control displays (at least control displays (C1) and (C2)) for the above sheet strip to be encased inside. The “detection display window” and “control display windows” may be provided separately, or together as a single window.

In a menopausal phase monitor 1 of the invention equipped with an above sheet strip, the sample application zone (A) is first impregnated with a test sample when used. Absorbed sample in the sample application zone (A) is then permeated through the sheet strip by capillary action, and proceed to the reaction zone (B) where detachable labeled antibodies [menopausal phase monitor 1 (FSH): labeled anti-FSH antibodies, menopausal phase monitor 1 (LH): labeled anti-LH antibodies] are positioned. In this zone, test substance (FSH or LH) in the sample specifically binds to an above labeled antibody by an antigen-antibody reaction [in the menopausal phase monitor 1 (FSH), FSH binds to labeled anti-FSH antibodies, and in the menopausal phase monitor 1 (LH), LH binds to labeled anti-LH antibodies]. Subsequently, the sample continues to migrate to the test result display (C0) of the detection area (C) together with “the labeled antibodies specifically bound to the test substance” (menopausal phase monitor 1 (FSH): complex between FSH and labeled anti-FSH antibodies, menopausal phase monitor 1 (LH): complex between LH and labeled anti-LH antibodies) and “excess labeled antibodies unbound to the test substance” (menopausal phase monitor 1 (FSH): labeled anti-FSH antibodies, menopausal phase monitor 1 (LH): labeled anti-LH antibodies). Since unlabeled antibodies (menopausal phase monitor 1 (FSH): unlabeled anti-FSH antibodies, menopausal phase monitor 1 (LH): unlabeled anti-LH antibodies) that can specifically bind to the test substance are stably immobilized at the test result display (C0), “labeled antibodies specifically bound to the test substance” in the sample are captured and accumulated in this place. Consequently, the test result display (C0) develops color at an intensity in accordance with the amount of test substance in the sample, based on the labeling agent of the captured labeled antibodies. Due to such a mechanism, presence/absence and quantitative proportions of the test substance in the sample can be detected.

The sample continues traveling to the control displays (C1) and (C2) of the detection area (C) together with “excess labeled antibodies unbound to the test substance”. Since secondary antibodies [menopausal phase monitor 1 (FSH): unlabeled anti-(anti-FSH antibody) antibodies, menopausal phase monitor 1 (LH): unlabeled anti-(anti-LH antibody) antibodies] that specifically bind to the labeled antibodies are stably immobilized at a given amount at the control displays (C1) and (C2), the above excess labeled antibodies in the sample are captured and accumulated at these displays. As a result, the control displays (C1) and (C2) develop color at intensities based on the amount of the secondary antibodies immobilized at each control display (C1) and (C2) (or the amount of the labeled antibodies bound to the secondary antibodies). If the amount of the secondary antibodies to be immobilized is predetermined at this stage, the relationship between the amount of labeled antibodies captured and the intensity of the developed color (tone) can be learned. Further, such a relationship becomes accurately known when the secondary antibody is immobilized in a different amount at each control displays (C1) and (C2). The menopausal phase monitor 1 (FSH/LH) shown on FIG. 2 as an embodiment of the invention has two control displays, however the invention is not limited to this embodiment and more than three control displays can be provided. More specifically, the amount of test substance in a test sample can be found by comparing the color intensities (tones) developed at at least two control displays with the color intensity (tone) developed at the above test result display.

This relationship is shown in Table 2 by way of an example using a blood sample. In the table, “<” indicates the order of intensity of color developed at each line [test result display (C0), and control displays (C1) and (C2)] on the detection area (C) of the monitor.

TABLE 2

| | Phase A | Phase B | Phase C | Phase D | Phase E |
|---|---|---|---|---|---|
| Hormone | FSH → | FSH ↑ | FSH ↑ | FSH ↑ | FSH ↑↑ |
| changes in | LH → | (Initial FP) | LH ↑ | LH ↑ | LH ↑↑ |
| blood | | LH → | | | |
| FSH | | | | | |
| Color | (c0) < (c1) < (c2) | (c1) < (c0) < (c2) | (c1) < (c0) < (c2) | (c1) < (c0) < (c2) | (c1) < (c2) < (c0) |
| tones at | | | | | |
| test | | | | | |
| result | | | | | |
| display | | | | | |
| (c0), | | | | | |
| control | | | | | |
| displays | | | | | |
| (c1) and | | | | | |
| (c2) | | | | | |
| LH | | | | | |
| Color | (c0′) < (c1′) < (c2′) | (c0′) < (c1′) < (c2′) | (c1′) < (c0′) < (c2′) | (c1′) < (c0′) < (c2′) | (c1′) < (c2′) < (c0′) |
| tones at | | | | | |
| test | | | | | |
| result | | | | | |
| display | | | | | |
| (c0′) and | | | | | |
| control | | | | | |
| displays | | | | | |
| (c1′) and | | | | | |
| (c2′) | | | | | |

FP: Follicular phase

For example, follicle stimulating hormone (FSH) levels in blood are known to fluctuate up to 15 mIU/mL in Phase A, from 15 to 30 mIU/mL in Phase B, from 15 to 30 mIU/mL in Phase C, 10 from 15 to 30 mIU/mL in Phase D, and be 30 mIU/mL or more in Phase E of the menopausal transition. Accordingly, subjects can identify their menopausal phase (Phase A, Phase B to D, or Phase E) using a menopausal phase monitor 1 (FSH) (sample: blood) comprising a detection area (C) provided with a control display (C1) at which anti-(anti-FSH antibody) antibodies (secondary antibodies) are immobilized so as to quantitatively detect anti-FSH antibodies in an amount that binds to 25 mIU/mL of FSH, and a control display (C2) at which anti-(anti-FSH antibody) antibodies (secondary antibodies) are immobilized so as to quantitatively detect anti-FSH antibodies in an amount that binds to 50 mIU/mL of FSH.

Further, luteinizing hormone (LH) levels in blood are known to fluctuate up to 25 mIU/mL in Phase A, to 25 mIU/mL in Phase B, from 25 to 50 mIU/mL in Phase C, from 25 to 50 mIU/mL in Phase D, and be 50 mIU/mL or more in Phase E of the menopausal transition. Accordingly, subjects can identify their menopausal phase (Phase A to B, Phase C to D, or Phase E) using a menopausal phase monitor 1 (LH) (sample: blood) comprising a detection area (C) provided with a control display (C1) at which anti-(anti-LH antibody) antibodies (secondary antibodies) are immobilized so as to quantitatively detect anti-LH antibodies in an amount that binds to 15 mIU/mL of LH, and a control display (C2) at which anti-(anti-LH antibody) antibodies (secondary antibodies) are immobilized so as to quantitatively detect anti-LH antibodies in an amount that binds to 30 mIU/mL of LH.

Furthermore, subjects can identify their menopausal phase (Phase A, Phase B, Phases C and D, or Phase E) using a menopausal phase monitor 1 (FSH/LH) (sample: blood) comprising a detection area (C) provided with a control display (c1) at which anti-(anti-FSH antibody) antibodies (secondary antibodies) are immobilized so as to quantitatively detect anti-FSH antibodies in an amount that binds to, e.g. 25 mIU/mL of FSH, and a control display (c1′) at which anti-(anti-LH antibody) antibodies (secondary antibodies) are immobilized so as to quantitatively detect anti-LH antibodies in an amount that binds to 15 mIU/mL of LH, as well as a control display (c2) at which anti-(anti-FSH antibody) antibodies (secondary antibodies) so as to quantitatively detect anti-FSH antibodies in an amount that binds to 50 mIU/mL of FSH, and a control display (c2′) at which anti-(anti-LH antibody) antibodies (secondary antibodies) so as to quantitatively detect anti-LH antibodies in an amount that binds to 30 mIU/mL of LH.

When a urine sample is used, about 2 to about 10% of each of the above values can be considered as guideline values (Neuroendocrinology; Shin Seirikagaku Taikei (New Physiological Science Series), Vol. 14, Igaku-Shoin Ltd., 1988, page 122).

(2) Type 2: Menopausal Phase Monitor

An embodiment of a Type 2 menopausal phase monitor is shown in FIG. 2.

The illustrated menopausal phase monitor 2 is adapted to detect two test substances, i.e. follicle stimulating hormone (FSH) and luteinizing hormone (LH), concurrently. A menopausal phase monitor 2 comprises a sheet strip 30 provided with a sample application zone (A), a reaction zone (B), a detection area (C) containing a test result display (C0) and control displays (Cn: where n is a positive number), and a sample collection zone (D). Menopausal phase monitor 2 is equipped with a support 10 for holding the sheet strip as are menopausal phase monitors 1.

The gross structure of the sheet strip 30 is the same as that of menopausal phase monitor 1 described earlier. However, the sheet strip for the menopausal phase monitor 2 is equipped with two sheets 32 and 33 aligned parallel to each other so as to form a gap 34 therebetween, with a reaction zone (B) and a detection area (C) being components of each of these sheets. The sheet strip is equipped with a sheet 31 that is a component of the sample application zone (A), sheets 32 and 33 that are components of the detection area (C), and a sheet 35 that is a component of the sample collection zone (D) in that order from upstream to downstream in the lengthways direction, and one end of each sheet 31 and 35 overlaps a different end of each of the sheets 32 and 33, thereby enabling a sample to migrate. The reaction zones (B) are located on the sheets 32 and 33 at a position close to the sheet 31.

More specifically, such menopausal phase monitors 2 are equipped with an FSH reaction site (b) carrying labeled anti-FSH antibodies and an LH reaction site (b′) carrying labeled anti-LH antibodies at the reaction zone (B) for simultaneous detection of two test substances, FSH and LH. Further, such menopausal phase monitor 2 comprises a detection area (C) provided with an FSH detection zone (c) having FSH control displays (c1) and (c2) each with different concentrations of unlabeled anti-(anti-FSH antibody) antibodies immobilized thereon, and an FSH test result display (c0) with unlabeled anti-FSH antibodies immobilized thereon disposed at intervals, and an LH detection zone (c′) having LH control displays (c1′) and (c2′) each with different concentrations of unlabeled anti-(anti-LH antibody) antibodies immobilized thereon,and an LH test result display (c0′) with unlabeled anti-LH antibodies immobilized thereon disposed at intervals.

As described above, FIG. 2 shows an example of a menopausal phase monitor 2 wherein the FSH reaction site (b) and the LH reaction site (b′), and the FSH detection zone (c) and the LH detection zone (c′) are separated by a gap. However, the arrangement of the FSH reaction site (b), LH reaction site (b′), FSH detection zone (c) and LH detection zone (c′) is not limited to the illustrated embodiment.

Using a menopausal phase monitor 2 which operates according to similar principles to those of menopausal phase monitor 1, a subject can detect the amount of test substances (FSH and LH) in a test sample, and thereby identifing the menopausal phase the subject is at. FIG. 2 incidentally shows a menopausal phase monitor 2 comprising control displays two each at two locations for detecting respective test substances (FSH and LH) [FSH control displays (c1) and (c2), and LH control displays (c1′) and (c2′)], however a menopausal phase monitor 2 may comprise a single control display for the detection of each test substance (FSH and LH). The detection principles and disposition of control displays are as described above.

For example, as described above, it is known that follicle stimulating hormone (FSH) levels in blood fluctuate up to 15 mIU/mL in Phase A, from 15 to 30 mIU/mL in Phase B, from 15 to 30 mIU/mL in Phase C, from 15 to 30 mIU/mL in Phase D, and are 30 mIU/mL or more in Phase E, and that luteinizing hormone (LH) levels in blood fluctuate up to 25 mIU/mL in Phase A, up to 25 mIU/mL in Phase B, from 25 to 50 mIU/mL in Phase C, from 25 to 50 mIU/mL in Phase D, and are 50 mIU/mL or more in Phase E of the menopausal transition.

Accordingly, with a menopausal phase monitor 2 equipped with a control display at each of FSH detection zone and LH detection zone, subjects can identify their menopausal phase (Phase A, Phase B, or Phase C to E), using a menopausal phase monitor 2 (sample: blood) comprising an FSH control display (c1) of an FSH detection zone (c) at which anti-(anti-FSH antibody) antibodies (secondary antibodies) are immobilized so as to quantitatively detect anti-FSH antibodies in an amount that binds to 25 mIU/mL of FSH, and an LH control display (c1′) of an LH detection zone (c′) at which anti-(anti-LH antibody) antibodies (secondary antibodies) are immobilized so as to quantitatively detect anti-LH antibodies in an amount that binds to 15 mIU/mL of LH. Further, subjects can identify their menopausal phase (Phase A to B, Phase C to D, or Phase E), using a menopausal phase monitor 2 (sample: blood) comprising an FSH control display (c1) of an FSH detection zone (c) at which anti-(anti-FSH antibody) antibodies (secondary antibodies) are immobilized so as to quantitatively detect anti-FSH antibodies in an amount that binds to, e.g. 50 mIU/mL FSH, and an LH control display (c1′) of the LH detection zone (c) at which anti-(anti-LH antibody) antibodies (secondary antibodies) are immobilized so as to quantitatively detect anti-LH antibodies in an amount that binds to 15 mIU/mL of LH. Furthermore, subjects can identify their menopausal phase (Phase A, Phase B to D, or Phase E), using a menopausal phase monitor 2 (sample: blood) comprising an FSH control display (cl) of an FSH detection zone (c) at which anti-(anti-FSH antibody) antibodies (secondary antibodies) are immobilized so as to quantitatively detect anti-FSH antibodies in an amount that binds to 25 mIU/mL of FSH, and an LH control display (c1′) of an LH detection zone (c′) at which anti-(anti-LH antibody) antibodies (secondary antibodies) are immobilized so as to quantitatively detect anti-LH antibodies in an amount that binds to 30 mIU/mL of LH. Moreover, subjects can identify their menopausal phase (Phase A to D, or Phase E), using a menopausal phase monitor 2 (sample: blood) comprising an FSH control display (cl) of an FSH detection zone (c) at which anti-(anti-FSH antibody) antibodies (secondary antibodies) are immobilized so as to quantitatively detect anti-FSH antibodies in an amount that binds to 50 mIU/mL of FSH, and an LH control display (c1′) of an LH detection zone (c′) at which anti-(anti-LH antibody) antibodies (secondary antibodies) are immobilized so as to quantitatively detect anti-LH antibodies in an amount that binds to 30 mIU/mL of LH.

As described above, menopausal phase monitors 2, as with Type 1 monitors, can also be equipped with two or more control displays [FSH control displays (c1) and (c2), and LH control displays (c1′) and (c2′)] at each detection zone [FSH detection zone (c) and LH detection zone (c′)]. With such a menopausal phase monitor 2 (sample: blood), subjects can identify their menopausal phase (Phase A, Phase B, Phase C to D, or Phase E), using a menopausal phase monitor 2 (sample: blood) comprising an FSH control display (c) equipped with an FSH control display (c1) at which anti-(anti-FSH antibody) antibodies (secondary antibodies) are immobilized so as to quantitatively detect anti-FSH antibodies in an amount that binds to 25 mIU/mL of FSH, and an FSH control display (c2) at which anti-(anti-FSH at antibody) antibodies (secondary antibodies) so as to quantitatively detect anti-FSH antibodies in an amount that binds to 50 mIU/mL of FSH, and an LH control display (c′) equipped with an LH control display (c1′) at which anti-(anti-LH antibody) antibodies (secondary antibodies) are immobilized so as to quantitatively detect anti-LH antibodies in an amount that binds to 15 mIU/mL of LH, and an LH control display (c2′) at which anti-(anti-LH antibody) antibodies (secondary antibodies) are immobilized so as to quantitatively detect anti-LH antibodies in an amount that binds to 30 mIU/mL of LH.

Menopausal phase monitors 2 are equipped with two reaction sites [(b) and (b′)] and detection zones [(c) and (c′)] for simultaneous detection of follicle stimulating hormone (FSH) and luteinizing hormone (LH). Such menopausal phase monitors 2 include those of the following configurations.

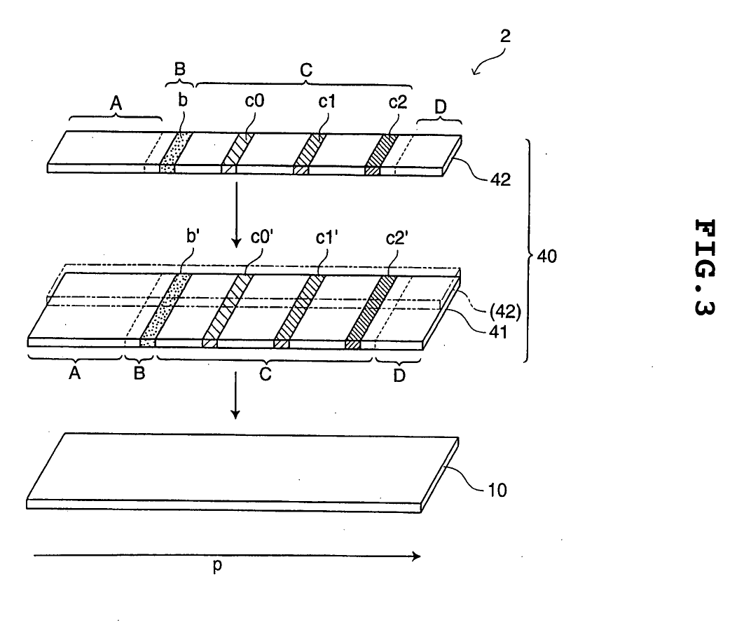

FIG. 3 shows a menopausal phase monitor 2 wherein a sheet strip 40, comprising two sheets 41 and 42 of unequal widths, is adhered to a support 10 (in the figure, these components are shown disassembled).

The sheet strip 40 has the sheet 42 with about half the width of the sheet 41 overlaying and adhered to the sheet 41 of about equal width to the support 10. A sample application zone (A), a reaction zone (B), a detection area (C), and a sample collection zone (D) are provided on the sheets 41 and 42 from one end to the other, enabling a test sample to migrate in that direction (shown by the arrow p in the figure).

In this embodiment, an FSH reaction site (b) carrying labeled anti-FSH antibodies, an FSH test result display (c0) with unlabeled anti-FSH antibodies immobilized thereon, and FSH control displays (cl) and (c2) with unlabeled anti-“anti-FSH antibody” antibodies immobilized thereon are provided on the wider sheet 41. Similarly, an LH reaction site (b′) carrying labeled anti-LH antibodies, an LH test result display (c0′) with unlabeled anti-LH antibodies immobilized thereon, and LH control displays (c1′) and (c2′) with unlabeled anti-“anti-LH antibody” antibodies immobilized thereon are provided on the narrower sheet 42. Alternatively, the wider sheet 41 may be equipped with the LH reaction site (b′), the LH test result display (c0′), the LH control displays (c1′) and (c2′), and the narrower sheet 42 may be equipped with the FSH reaction site (b), the FSH test result display (c0), and the FSH control displays (c1) and (c2). The sample application zone (A) and the sample collection zone (D) are provided on one end and the other end of the sheets 41 and 42 respectively in the lengthways direction.

The sheets 41 and 42 can be made of materials exemplified earlier, and they may be made of the same material or different materials. Examples of adhesives for the attachment of these sheets include alkyl acrylate copolymers, ethylene-vinyl acetate copolymers, alkyl acrylate-vinyl acetate copolymers, styrene-isopropylene-styreneblock copolymers, styrene-ethylenepropylene-styreneblock copolymers, styrene-butadiene-styreneblock copolymers, styrene-ethylenebutylene-styreneblock copolymers, noncrystalline polypropylene resins, noncrystalline propylene-ethylene copolymers, noncrystalline propylene-ethylene-butene copolymers, noncrystalline propylene-butene copolymers, etc.

In the sheet strip 40 comprising two sheets, 41 and 42, a test sample received at the sample application zone (A) mainly travels along each sheet separately, and thereby the test substance (FSH) bound to labeled anti-FSH antibodies and the other test substance (LH) bound to labeled anti-LH antibodies barely mix with each other between these two sheets, causing no problems in detection. In particular, an adhesion layer between the two sheets ensures such mixing problems are much less likely to occur.

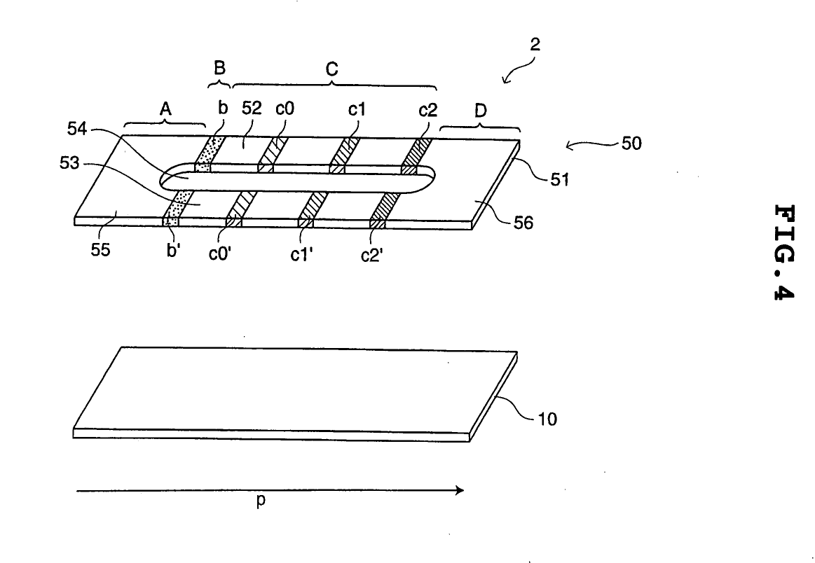

FIG. 4 shows a menopausal phase monitor 2 comprising a sheet strip 50 comprising a single sheet 51 with a gap provided in its center and adhered to a support 10 (in the figure, these components are shown disassembled).

The sheet 51 is about the same width as the support 10, with a gap 54 formed in its center extending in the lengthways direction of the sheet. A sample application zone (A), a reaction zone (B), a detection area (C) and a sample collection zone (D) are provided on the sheet 51 from one end to the other, enabling a sample to migrate in that direction (shown by the arrow p in the figure).

In this embodiment, on one side 52 of the sheet 51 partitioned by the gap 54 are provided an FSH reaction site (b) carrying labeled anti-FSH antibodies, an FSH test result display (c0) with unlabeled anti-FSH antibodies immobilized thereon, and FSH control displays (c1) and (c2) with unlabeled anti-(anti-FSH antibody) antibodies immobilized thereon. Further, on the other side 53 are provided an LH reaction site (b′) carrying labeled anti-LH antibodies, an LH test result display (c0′) with unlabeled anti-LH antibodies immobilized thereon, and LH control displays (c1′) and (c2′) with unlabeled anti-(anti-LH antibody) antibodies immobilized thereon. The sample application zone (A) and the sample collection zone (D) are provided at one end 55 and the other end 56 respectively in the lengthways direction of the sheet 51, where there is no gap 54.

The sheet 51 can be made of materials as exemplified earlier. With the simple structure provided by the gap 54, the sheet strip 50 can advantageously prevent a sample received at the sample application zone (A) from mixing after it is bound to labeled antibodies (labeled anti-(FSH) antibodies and labeled anti-(LH) antibodies) and to other antibodies.

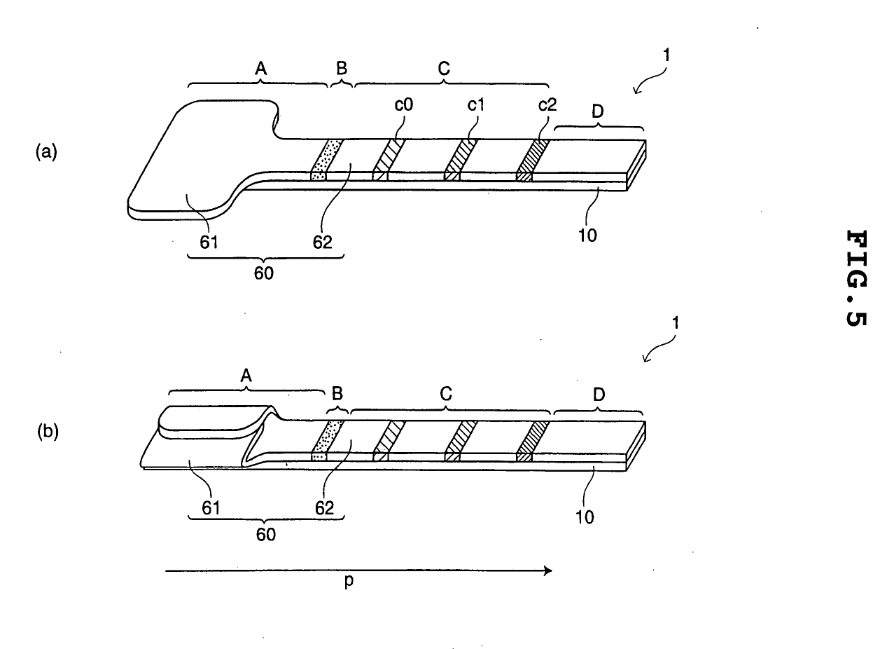

FIG. 5 shows an embodiment of a Type 1 menopausal phase monitor. This menopausal phase monitor 1 has a sheet strip 60 adhered to a support 10. The sheet strip 60 is, as described in earlier embodiments, equipped with a sample application zone (A), a reaction zone (B), a detection area (C) and a sample collection zone (D) from one end to the other end in the lengthways direction in the order mentioned. In this embodiment, as shown in FIG. 5(a), the sample application zone (A) is wider in the width direction than are the other zones and area, and as shown in FIG. 5(b), both sides of the wider portion in the width direction are folded into the center until use. The sheet strip 60 can be made of the materials exemplified earlier.

With the menopausal phase monitor 1 shown in FIG. 5, the sample application zone (A) is folded until use, and the monitor is hence conveniently packed and carried due to its small size. Further, the sample application zone (A) is unfolded widthways for use, whereby receiving a sample easily and securely with its large area. In particular, when a urine sample is used, it is advantageous for easy sample collection on urination. Such a wider sample application zone (A) can also be used for Type 2 menopausal phase monitors (e.g. the embodiments shown in FIGS. 2 to 4).

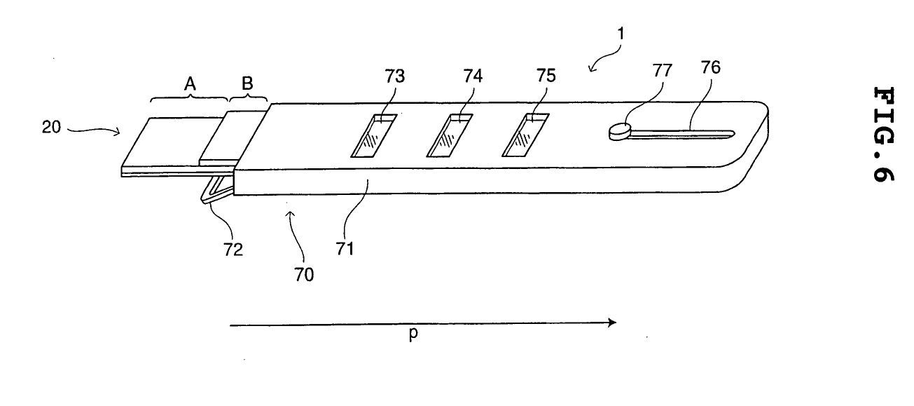

FIG. 6 shows an embodiment of a Type 1 menopausal phase monitor with a sheet strip 20 accommodated in a case. In this menopausal phase monitor 1, the sheet strip 20 shown in FIG. 1 is accommodated in an elongated case 70 so that the sheet tip can be easily pushed in and out. The case 70 is equipped with a body 71 having an opening at one end of the lengthways direction, and a lid 72 fixed to the opening by means of a hinge. At the other end of the case, a slot 76 extending in the lengthways direction is provided with a button 77 slidable therein. The interior end of the button 77 is fixed to the end of the sheet strip 20. Accordingly, when the button 77 is slid toward one end of the case 20, the sheet strip 20 is pulled inside the case 70, and when the button is slid toward the opposite end, the tip of the sheet strip 20 can be exposed from the case 70 as shown in the figure. The case 70 is provided with windows 73, 74, and 75 corresponding to the positions of the test result display (C0) and the control displays (C1) and (C2) respectively when the tip of the sheet strip 20 is pushed out. These windows are preferably covered with a translucent plastic plate or the like.

In this menopausal phase monitor 1, the sheet strip 20 is pulled into the case 70, and the lid 72 is closed for packing and carrying before use, and when used, the lid 72 is opened and the button 77 is slid to expose the tip of the sheet strip 20 (sample application zone (A)). At this position, a sample can be brought into contact with the application tip. After use, the sheet strip 20 can be withdrawn and the lid 72 closed. As described, this menopausal phase monitor 1 is easy to operate, and when the sheet strip is packed and carried, it can be kept clean before and after a sample is applied.

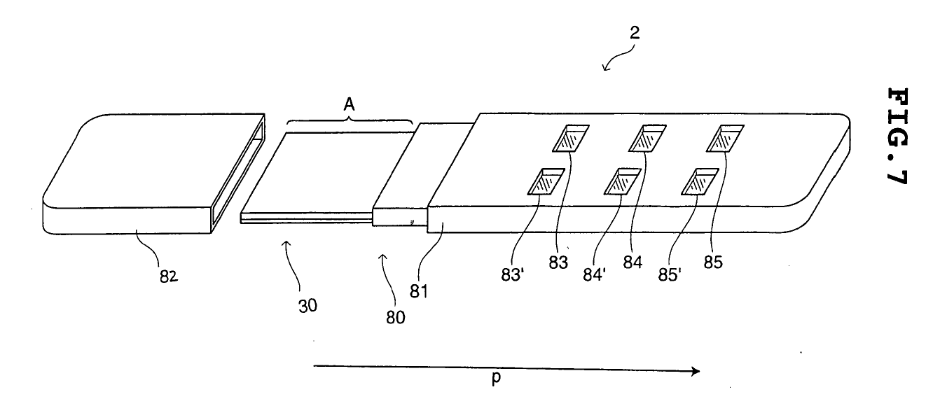

FIG. 7 shows an embodiment of a Type 2 menopausal phase monitor with a sheet strip accommodated in a case. This menopausal phase monitor has the sheet strip 30 shown in FIG. 2 accommodated in an elongated case 80. The case 80 is equipped with a case body 81 having an opening at one end of the lengthways direction and a cap 82 removably placed at the opening. When the cap 82 is removed, the tip of the sheet strip 30 can be exposed from the case body 81 as shown in the figure. The case body 81 is provided with windows 83, 84, and 85 corresponding to the positions of an FSH test result display (c0) and FSH control displays (c1) and (c2) for a test substance FSH respectively, and is also provided with windows 83′, 84′, and 85′ corresponding to positions for an LH test result display (c0′) and LH control displays (c1′) and (c2′) for a test substance LH, respectively. These windows are preferably covered with a translucent plastic plate or the like. Alternatively, at least some of such a plurality of windows can be connected in the lengthways direction or widthways direction of the case body 81. When only one control display is provided for each test substance, windows corresponding thereto are provided.

In this menopausal phase monitor 2, the sheet strip 30 is accommodated in the case body 81 with the cap 82 closed for packing and carrying before use, and for use, the cap 82 is removed to expose the tip of the sheet strip 30 (sample application zone (A)). At this position, a sample can be brought into contact with the tip. After use, the cap 82 is repositioned at the case body 81 to enclose the sheet strip 30. As described, this menopausal phase monitor 2 is also easy to operate, and when the sheet strip is packed and carried, it can be kept clean before and after a sample is applied.

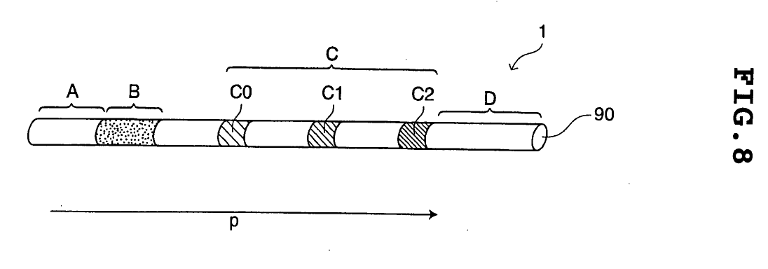

In menopausal phase monitors 1 and 2, absorbent members of various configurations can be used in place of the aforementioned sheet strips. FIGS. 8 to 10 show embodiments using rods as absorbent members.

FIG. 8 shows a menopausal phase monitor 1 provided with a rod 90 in a circular cylindrical configuration. The menopausal phase monitor is equipped with a sample application zone (A), a reaction zone (B), a detection area (C) and a sample collection zone (D) in that order from one end to the other end in the lengthways direction of the rod 90.

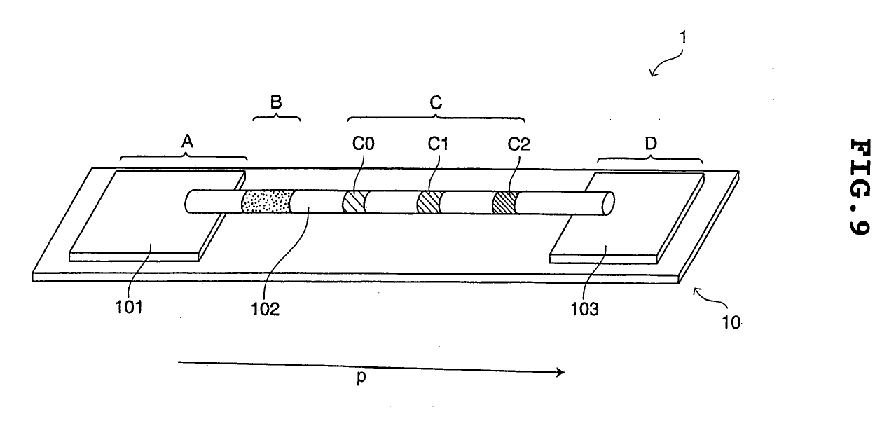

FIG. 9 shows a menopausal phase monitor 1 comprising an absorbent member with sheet members 101 and 103 disposed at one of each end of a circular cylindrical rod member 102. These components can be joined by means of adhesives, strings, wires, heat-sealing, etc. in such a manner that the sample migration properties between each member are maintained. The menopausal phase monitor 1 is provided with a sheet member 101 as a sample application zone (A), a rod member 102 as a reaction zone (B) and a detection area (C), and a sheet member 103 as a sample collection zone (D). The absorbent member comprising these components is further fixed to a support 10 by adhesive, etc.

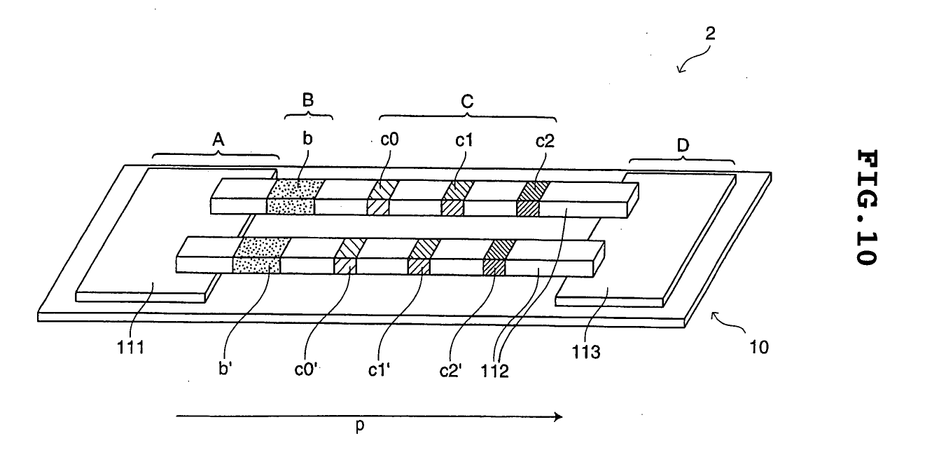

FIG. 10 shows a menopausal phase monitor 2 provided with an absorbent member comprising rectangular cylindrical rods, and sheet members 111 and 113 respectively disposed at the ends of the rods. These components can also be joined by means of adhesive, string, wire, heat-sealing, etc. in such a manner that the sample migration properties between the members are maintained. The menopausal phase monitor 2 is provided with a sheet member 111 as a sample application zone (A), one of the two rod members 112 as a reaction site (b) and a detection zone (c) for FSH [FSH test result display (c0) and FSH control displays (c1) and (c2)], the other rod member as a reaction site (b′) and a detection zone (c′) [LH test result display (c0′) and LH control displays (c1′) and (c2′)], and a sheet member 113 as a sample collection zone (D). The absorbent member comprising these components is further fixed to a support 10 by means of adhesive, etc. Short narrow menopausal phase monitors can be obtained with such absorbent members using these rod members. In particular, these absorbent members can be advantageously used for Type 2 menopausal phase monitors. Such menopausal phase monitors 2 can also be enclosed in cases as shown in FIGS. 6 and 7. Further, windows provided in the cases can be convex lenses for easy visibility of the colors developed on the detection area.

A written descriptive means (specifications) such as an instruction sheet can accompany for these menopausal phase monitors 1 and 2 of the invention along with an instruction sheet explaining how to identify menopausal phases, and thereby the present invention provides a menopausal phase monitor kit containing a set of such a menopausal phase monitor 1 or 2 and such written descriptive means (specifications). Examples of an instruction sheet explaining how to identify menopausal phases include sheets specifically stating what intensities (tones) of the developed colors appearing on test result display and control displays mean in identifying menopausal phases (Phases A to E). Such descriptive statements can be in the form of specifications sheets, or displays indicated on the case surfaces shown in FIGS. 6 and 7.

In addition to the embodiments described above, Types 1 and 2 menopausal phase monitors can be embodied in various other configurations without departing from the scope of the present invention.

1. A menopausal phase monitor provided with an absorbent member composed of a material capable of migrating a test sample by capillary action, wherein the absorbent member comprises:
(1a) a sample application zone for receiving and absorbing a test sample;
(1b) an FSH reaction site carrying labeled anti-FSH antibody specifically reactive with follicle stimulating hormone (FSH);
(1c) an FSH detection zone equipped with at least two FSH control displays each with a different amount of immobilized antibody, and an FSH test result display disposed at intervals,
(1) the FSH test result display having unlabeled anti-FSH antibody specifically reactive with follicle stimulating hormone (FSH) immobilized thereon, and
(2) the FSH control displays having unlabeled anti-(anti-FSH antibody) antibody specifically reactive with anti-FSH antibody immobilized thereon; and
(1d) a sample collection zone for collecting residual sample that has migrated from the sample application zone, the FSH reaction site, and the FSH detection zone.

2. A menopausal phase monitor provided with an absorbent member composed of a material capable of migrating a test sample by capillary action, wherein the absorbent member comprises:
(2a) a sample application zone for receiving and absorbing a test sample;
(2b) an LH reaction site carrying labeled anti-LH antibody specifically reactive with luteinizing hormone (LH);
(2c) an LH detection zone equipped with at least two LH control displays with a different amount of immobilized antibody, and an LH test result display disposed at intervals,
(1) the LH test result display having unlabeled anti-LH antibody specifically reactive with luteinizing hormone (LH) immobilized thereon, and
(2) the LH control displays having unlabeled anti-(anti-LH antibody) antibody specifically reactive with anti-LH antibody immobilized thereon; and
(2d) a sample collection zone for collecting residual sample that has migrated from the sample application zone, the LH reaction site, and the LH detection zone.

3. A menopausal phase monitor provided with an absorbent member composed of a material capable of migrating a sample by capillary action, wherein the absorbent member comprises:
(3a) a sample application zone for receiving and absorbing a sample;
(3b) a reaction zone comprising an FSH reaction site carrying labeled anti-FSH antibody specifically reactive with follicle stimulating hormone (FSH), and an LH reaction site carrying labeled anti-LH antibody specifically reactive with luteinizing hormone (LH);
(3c) a detection area comprising an FSH detection zone and an LH detection zone,
(3c-1) the FSH detection zone being equipped with at least two FSH control displays each with a different amount of immobilized antibody, and an FSH test result display disposed at intervals,
(i) the FSH test result display having unlabeled anti-FSH antibody specifically reactive with follicle stimulating hormone (FSH) immobilized thereon,
(ii) the FSH control displays having unlabeled anti-(anti-FSH antibody) antibody specifically reactive with anti-FSH antibody immobilized thereon,
(3c-2) the LH detection zone being equipped with at least two LH control displays each with a different amount of immobilized antibody, and an LH test result display disposed at intervals,
(i) the LH test result display having unlabeled anti-LH antibody specifically reactive with luteinizing hormone (LH) immobilized thereon, and
(ii) the LH control displays having unlabeled anti-(anti-LH antibody) antibody specifically reactive with anti-LH antibody immobilized thereon; and
(3d) a sample collection zone for collecting residual sample that has migrated from the sample application zone, the reaction zone, and detection area.

4. A menopausal phase monitor provided with an absorbent member composed of a material capable of migrating a test sample by capillary action, wherein the absorbent member comprises:
(4a) a sample application zone for receiving and absorbing a test sample;
(4b) an FSH reaction site carrying labeled anti-FSH antibody specifically reactive with follicle stimulating hormone (FSH), and an LH reaction site carrying labeled anti-LH antibody specifically reactive with luteinizing hormone (LH);
(4c-1) an FSH detection zone equipped with an FSH control display, and an FSH test result display disposed at intervals,
(1-1) the FSH test result display having unlabeled anti-FSH antibody specifically reactive with follicle stimulating hormone (FSH) immobilized thereon, and
(1-2) the FSH control display having unlabeled anti-(anti-FSH antibody) antibody specifically reactive with anti-FSH antibody immobilized thereon;
(4c-2) an LH detection zone equipped with an LH control display, and an LH test result display disposed at intervals,
(2-1) the LH test result display having unlabeled anti-LH antibody specifically reactive with LH immobilized thereon, and
(2-2) the LH control display having unlabeled anti-(anti-LH antibody) antibody specifically reactive with anti-LH antibody immobilized thereon; and
(4d) a sample collection zone for collecting residual sample that has migrated from the sample application zone, the FSH reaction site, the LH reaction site, the FSH detection zone, and the LH detection zone.

5. A menopausal phase monitor according to claim 4, wherein the FSH detection zone comprises an FSH test result display and at least two FSH control displays each with different concentrations of immobilized unlabeled anti-(anti-FSH antibody) antibody, and the LH detection zone comprises an LH test result display and at least two LH control displays each with different concentrations of immobilized unlabeled anti-(anti-LH antibody) antibody.

6. A menopausal phase monitor provided with an absorbent member composed of a material capable of migrating a test sample by capillary action, wherein the absorbent member comprises:
(5a) a sample application zone for receiving and absorbing a test sample;
(5b) an FSH reaction site carrying labeled anti-FSH antibody specifically reactive with follicle stimulating hormone (FSH), and an LH reaction site carrying labeled anti-LH antibody specifically reactive with luteinizing hormone (LH);
(5c-1) an FSH detection zone equipped with at least two FSH control displays each with a different amount of immobilized antibody, and an FSH test result display disposed at intervals,
(1-1) the FSH test result display having unlabeled anti-FSH antibody specifically reactive with follicle stimulating hormone (FSH) immobilized thereon, and
(1-2) the FSH control displays having unlabeled anti-(anti-FSH antibody) antibody specifically reactive with anti-FSH antibody immobilized thereon;
(5c-2) an LH detection zone equipped with at least two LH control displays each with a different amount of immobilized antibody, and an LH test result display disposed at intervals,
(2-1) the LH test result display having unlabeled anti-LH antibody specifically reactive with luteinizing hormone (LH) immobilized thereon, and
(2-2) the LH control displays having unlabeled anti-(anti-LH antibody) antibody specifically reactive with anti-LH antibody immobilized thereon; and
(5d) a sample collection zone for collecting residual sample that has migrated from the sample application zone, the FSH reaction site, the LH reaction site, the FSH detection zone, and the LH detection zone.

7. A menopausal phase monitor according to any one of claims 1 to 4 and 6, wherein the sample is female urine.

8. A menopausal phase monitor kit comprising a menopausal phase monitor according to any one of claims 1 to 4 and 6, and instructions on how to use the monitor and how to identify menopausal phases.
